(19) United States
(12) Patent Application Publication
(10) Pub. No.: US 2006/0280762 A1
(43) Pub. Date: Dec. 14, 2006

(54) Color Cosmetics - Coloring/Staining the skin from fruit and plant color pigments

(75) Inventors: Richard Hughes Kostick, Oakland, CA (US); Susie Pei Hwa Wang, Oakland, CA (US); James Pei Fung Wang, Oakland, CA (US)

Correspondence Address: Richard Hughes Kostick, 6839 Elverton Drive, Oakland, CA, 94611, US

(73) Assignees: Richard Hughes Kostick, Oakland, CA (US); Susie Pei Hwa Wang, Oakland, CA (US); James Pei Fung Wang, Oakland, CA (US)

(21) Appl. No.: 10/908,909

(22) Filed: May 31, 2005

(51) Int. Cl. A61K 6/00

(52) U.S. Cl. 424/401

(57) ABSTRACT

The use of color pigment from fruits and vegetables for cosmetic purposes of coloring the skin such as staining lips, flushing cheeks and enhancing the eyes. These pigments can be used in lip sticks, lip stains, cheek stains, blush, eye liner, eye shadow, mascara, eyebrow powder/pencil, sheer to full coverage foundation, bronzer, highlighter. Color pigments can be found in fruits and vegetables in the form of carotenoids, chlorophyll, anthocyanin and anthoxanthin among others. Previously these colors have been used only to color cosmetic products themselves, not for coloring the skin.

The present invention can relate to a product, for example a cosmetic and/or dermatological product, comprising of the color pigment of fruits and/or vegetable for the expressed purpose of coloring/staining the skin. Color pigments of fruits and/or vegetables have been used to alter the color of cosmetic and dermatological products themselves, but never until now to alter the color and to stain the skin when applied. For example—pomegranate pigments staining the lips and cheeks.

Generally, Fruits and Vegetables are made up of water, CHO, fat and lipids, proteins, Amino Acids, vitamins, minerals, P, K, Ca, Mg, Fe, organic acids, pectins, pectic enzymes, pigments (including tannins), and odorous compounds

Pigments found in fruits and vegetables give color such as carotenoids, chlorophylls, anthocyanins, proanthcyanins, and anthoxanthins. These are the natural color pigments that stains/color to the skin when the pigment is applied on the skin.

Products that can use the pigments include lipsticks, lip stains, cheek stains, blush, eye makeup such as eye liner, eye shadow, mascara, eyebrow powder/pencil, sheer to full coverage foundation, bronzer, highlighter among other cosmetic and dermatological products.

The use of the color pigment from fruits and vegetables for cosmetic and dermatological purposes such as staining lips, flushing cheeks, enhancing eyes, which eliminates the use of synthetic colorants such as FD&C colors which traditional color cosmetics are made with. Previously, pigments have been from coal or insects and minerals.

The fruit or vegetable in its natural state can be used by rubbing it onto the skin or the fruit pigment can be extracted to color or stain the skin directly or can be added into a base of traditional cosmetic products (with or without the aid of FD&C colors) for easier spreadability. For example, we can create a traditional lipstick base of waxes and oils without the use of FD&C colors and using the fruit pigments to stain the lips

The fruit or vegetable can be dried into a powder and then added into formulas

The fruit or vegetable can be in an extracted state and the pigment taken from an extract of the fruit

The fruit or vegetable can be added to any already existing formula or new formula. No special technique is needed to use the new type of skin coloring pigment

The benefits of using fruit or vegetable pigments go beyond coloring/enhancing/staining the skin to also delivering vitamins and antioxidants

What is claimed is:

1. A dermatological or cosmetic product for coloring/staining the skin using a plant or vegetable pigment.

2. The product in accordance with claim 1 wherein said plant or vegetable pigment is about 0.0001% to 100% by weight of said composition.

3. A product in accordance with claims 1 and 2 wherein the skin is colored immediately or over a period of time.

4. A product in accordance with claims 1 and 2 wherein the color pigment is extracted from the fruit or vegetable.

5. A product in accordance with claims 1 and 2 wherein the fruit or vegetable is added to the product in part or whole wherein the color pigment is included in the part or whole.

6. A product in accordance with claims 1, 2 and 5 wherein the fruit or vegetable pigment is added to the product in a powder form, liquid form, gas form, or semi-liquid form.
